(19) United States
(12) Patent Application Publication
(10) Pub. No.: US 2009/0030338 A1
(43) Pub. Date: Jan. 29, 2009

(54) ACCESS GUIDE AND METHODS OF USING THE SAME

(75) Inventors: Michael D. Crocker, Half Moon Bay, CA (US); Steve Trebotich, Newark, CA (US); Tom Brockman, Hayward, CA (US); Daniel Kraft, Stanford, CA (US)

Correspondence Address: LEVINE BAGADE HAN LLP, 2483 EAST BAYSHORE ROAD, SUITE 100, PALO ALTO, CA, 94303, US

(73) Assignee: StemCor Systems, Inc., Menlo Park, CA (US)

(21) Appl. No.: 11/828,048

(22) Filed: Jul. 25, 2007

(51) Int. Cl. A61B 10/02

(52) U.S. Cl. 600/562

(57) ABSTRACT

Devices and methods for guiding surgical tools are disclosed. The guided surgical tools can be used to extract body tissue from an enclosed body cavity. The guide can have multiple channels for the surgical tools to pass through the guide. The channels can converge and exit at a single exit port. The channels can have distinct entry ports. The guide can have a configuration to provide stable seating at or adjacent to the target site.

### BACKGROUND OF THE INVENTION

i. Field of the Invention

The invention relates to devices and methods for guiding a tissue extracting device into and within an enclosed body cavity.

ii. State of the Related Art

Bone Marrow is a rich source of pluripotent hematopoietic stem cells from which red blood cells, white blood cells, and platelets are formed. Bone marrow also contains additional populations of mesenchymal stem cells and other stem and progenitor cells which have the potential to repair and regenerate other tissues.

Since the early 1970's bone marrow and hematopoietic stem cell transplantation has been used to treat patients with a wide variety of disorders, including but not limited to cancer, genetic and autoimmune diseases. Currently over 60,000 transplants for a variety of indications are performed worldwide each year.

In autologous transplants, the patient has their own bone marrow collected prior to receiving high dose chemotherapy. Following high dose, myeloablative chemotherapy, which kills the majority of the patients' marrow stem cells, the stored autologous marrow or hematopoietic stem cells purified or enriched from the marrow are infused, and serves to improve the patient's hematolymphoid system.

In allogeneic transplants bone marrow, or other sources of hematopoietic stem cells derived from a full or partially human leukocyte antigen (HLA) matched sibling, parent or unrelated donor is infused into the recipient patient and following engraftment, serves to reconstitute the recipients hematopoietic system with cells derived from the donor.

Following myeloablative or non-myeloablative conditioning of a patient with chemotherapy and/or radiation therapy, the marrow is regenerated through the administration and engraftment of hematopoietic stem cells contained in the donor bone marrow.

In addition to hematopoietic stem cells and hematopoietic progenitors, bone marrow contains mesenchymal and other stem cell populations thought to have the ability to differentiate into muscle, myocardium, vasculature and neural tissues and possibly some organ tissues such as liver and pancreas. Research in preclinical animal studies and clinical trials suggest that bone marrow or some portion of the cells contained within marrow can regenerate tissues other than the hematopoietic system. This includes the ability for cells contained within the marrow to regenerate or facilitate repair of myocardial tissue following a myocardial infarction, and in the setting of congestive heart failure as evident by improved cardiac function and patient survival.

Bone marrow derived stem cells also show evidence for their ability to regenerate damaged liver and hepatic cells and portions of the nervous system including spinal cord. Additional organ systems including kidney and pancreas show benefit from bone marrow derived cells. Use of bone marrow and the stem cells contained within bone marrow may be of increasing clinical utility in the future treatment of patients. Furthermore a patient's own marrow has multiple applications in orthopedic procedures, including but not limited to spinal fusions, treatment of non-union fractures, osteonecrosis, and tissue engineering.

Stem cells utilized in transplantation are usually collected using one of two methods. In a first method known as a bone marrow harvest, bone marrow is directly accessed in and removed from the patient usually by multiple aspirations of marrow from the posterior ileac crest. The bone marrow harvest procedure is often performed in the operating room.

To perform a harvest of 500-1500 milliliters of marrow, multiple separate entries into the marrow cavity are required to in order to remove a sufficient amount of bone marrow. A bone marrow aspiration needle, such as a sharp metal trocar, is placed into the marrow space through the soft tissue and the outer cortex of the ileac crest. The aspiration needle enters less than 2 cm into the marrow cavity. Negative pressure is applied through the hollow harvest needle, usually by the operator pulling on an attached syringe into which 5-10 ml of marrow is aspirated. The needle and syringe are then removed.

After removing the collected marrow, the aspiration needle accesses a separate location on the ileac bone for another aspiration. This method of inserting the needle into the bone, removing the marrow, and removing the needle from the bone is performed on the order of 100-300 separate entries for an average patient to remove a volume of bone marrow required for transplantation.

Each puncture and entry into the marrow cavity accesses only a limited area of the marrow space, and the majority of practitioners only remove 5-10 milliliters of marrow with each marrow penetration. Pulling more marrow from a single marrow entry site otherwise results in a collected sample highly diluted by peripheral blood.

The bone marrow harvest procedure requires general anesthesia because the ileac crest is penetrated 100-300 times with a sharp bone marrow trocar. Local anesthesia is generally not possible given the large surface area and number of bone punctures required.

The donor needs time to recover from general anesthesia, and frequently suffers from days of sore throat, a result of the endotracheal intubation tube placed in the operating room.

Pre-operative preparation, the harvest procedure, recovery from anesthesia, and an overnight observation stay in the hospital following the procedure requires considerable time on behalf of the donor and the physician, and similarly additional expense. The cost of the procedure is often $10,000 to $15,000, which includes costs for operating room time, anesthesia supplies and professional fees, and post-operative care and recovery.

In addition to general operating room staff, the traditional bone marrow harvest procedure requires two transplant physicians. Each physician aspirates marrow from the left or right side of the ileac crest. The procedure itself usually takes approximately one and half hours for each operating physician.

Many donors experience significant pain at the site of the multiple bone punctures which persists for days to weeks.

Traditional bone marrow aspiration incurs a significant degree of contamination with peripheral blood. Peripheral blood contains high numbers of mature T-cells unlike pure bone marrow. T-cells contribute to the clinical phenomenon termed Graft vs. Host Disease (GVHD), in both acute and chronic forms following transplant in which donor T-cells present in the transplant graft react against the recipient (host) tissues. GVHD incurs a high degree of morbidity and mortality in allogeneic transplants recipients.

In a second method to collect stem cells for transplantation, mononuclear cells are removed from the donor's peripheral blood. The peripheral blood contains a fraction of hematopoietic stem cells as well as other populations of cells including high numbers of T-cells. In this procedure peripheral blood stem cells are collected by apheresis following donor treatment with either chemotherapy—usually cyclophosphamide or with the cytokine Granulocyte Colony Stimulating Factor (GCSF). Treatment with cyclophosphamide or GCSF functions to mobilize and increase the numbers of hematopoietic stem cells circulating in the blood.

This collection method can be slow and time consuming. It requires the donor to first undergo five or more days of daily subcutaneous injections with high doses of the cytokine GCSF prior to the collection. These daily injections can be uncomfortable and painful and bone pain is a common side effect. Peripheral blood stem cells can not be obtained without this seven-plus day lead time.

Each day of apheresis costs approximately $3,000 including but not limited to the cost of the apheresis machine, nursing, disposable supplies and product processing. The patient often has to come back on multiple days in order to obtain an adequate number of stem cells. Costs for the GCSF drug alone approximate $6,000-$10,000 depending upon the weight of the patient.

Given the multiple days required to collect adequate numbers of hematopoietic stem cells, individual bags of peripheral blood product must be processed and frozen separately. These bags are then thawed, and given back to the recipient patient at the time of transplant. The volume, and chemicals contained in the product freezing media can cause some complications, such as mild side effects, at the time of infusion.

Accordingly, there is a need for a minimally invasive, less expensive, time-efficient bone marrow harvest procedure with minimal complications which does not require general anesthesia, offers fast recovery time, and does not cause significant pain to the bone marrow donor.

### SUMMARY OF THE INVENTION

An access guide and a method of using the same are disclosed. The guide can be used to direct one or more devices for manipulation and extraction of body tissue from an enclosed body cavity. For example, the guide can permit multiple aspirations of cancellous bone marrow at different angles through a single entry hole in the cortical bone. The guide can also have multiple extraction channels that approach a common exit port at a variety of angles.

In one variation of a method for use, the guide can be placed adjacent to the bone targeted for harvesting bone marrow. An access channel can be formed through the cortical bone adjacent to the exit port of the guide. For example, a sharpened trocar can be inserted through a first aspiration channel. The trocar can then be forced through the soft tissue and the cortical bone. The trocar can have a hollow longitudinal trocar channel that can act as a passageway from outside the guide to inside the cancellous bone cavity.

An aspiration tool, such as an aspiration cannula, can then be inserted through the trocar channel in the first aspiration channel, through the exit port of the guide, through the cortical access channel, and into the cancellous bone marrow. The bone marrow can then be extracted by the aspiration tool.

The trocar and the aspiration tool can then be removed from the bone and the guide. The trocar can then be re-inserted into the same guide through a second aspiration channel and again through the soft tissue and cortical bone. Inserting the trocar through the soft tissue and cortical bone may or may not remove any additional soft tissue or cortical bone since the trocar may or may not be pushed through the access channel previously formed in the soft tissue and the cortical bone when the trocar was directed through the first aspiration channel.

The aspiration tool can then be re-inserted through the trocar channel in the second aspiration channel. The second aspiration channel can be at a different angle of approach to the bone than the angle of approach of the first aspiration channel. The aspiration tool can then be inserted through the exit port of the guide, through the cortical access channel, and into the cancellous bone marrow. The aspiration tool can then aspirate additional marrow from the bone. Aspiration can be performed sequentially and/or concurrently with irrigation of the cancellous bone and/or disruption of the bone matrix, for example by rotation and/or translation of a whisk through the cancellous bone marrow.

In one variation of use, soft tissue can be cut and retracted away from the bone before the guide is placed on the bone. In another variation of use, soft tissue can remain un-cut and/or unretracted, and the guide can be placed firmly on the skin. In yet another variation of use, the guide can fixed to the bone, for example using fixation pins that pass through the guide and into the cortical bone.

A variation of the guide can have multiple aspiration channels, e.g., four or more aspiration channels. A variation of use includes using a solid bore to create or augment the access channel in the soft tissue and/or cortical bone.

The device and method may be used on any bone (which can be in vivo or in vitro), for example at the ileac crest or elsewhere on the pelvis, femur, humerus, other bone, or combinations thereof is disclosed.

### BRIEF DESCRIPTION OF THE DRAWINGS

FIG. 1 is a perspective view of a variation of the guide.

FIGS. 2a, 2b and 2c are front, side and top views, respectively, of a variation of the guide of FIG. 1.

FIG. 2d illustrates a side view of another variation where each access channel has a separate exit port.

FIGS. 3a, 3b and 3c are front, side and top views, respectively, of a variation of the guide of FIG. 1.

FIG. 4a illustrates a variation of a method for positioning the guide adjacent to the target bone with the body shown in see-through view.

FIG. 4b illustrates an example where the access channels having different angles may be used to guide an aspiration device within a bone cavity at different corresponding angles.

FIGS. 5a, 5b and 5c are front, side and top close-up views, respectively, of a variation of the placement of the guide as shown in FIG. 4.

FIGS. 6a, 6b and 6c are front, side and top close-up views, respectively, of a variation of a method for drilling a cortical access channel.

FIGS. 7a, 7b and 7c are front, side and top close-up views, respectively, of a variation of a method for deploying a trocar through the guide.

FIGS. 8a, 8b and 8c are front, side and top close-up views, respectively, of a variation of a method for extracting cancellous bone marrow.

FIG. 9 illustrates an assembled, partially schematic view of a variation of the aspiration device inserted through the trocar.

FIGS. 10 and 12 are side views of variations of the distal tip of the aspiration cannula.

FIG. 11 is a front view of a variation of the distal tip of FIG. 10.

FIGS. 13 and 14 are front views of variations of the distal tip of FIG. 12.

FIGS. 15a, 15b and 15c are front, side and top close-up views, respectively, of a variation of a method for extracting cancellous bone marrow.

FIGS. 16a, 16b and 16c are front, side and top close-up views, respectively, of a variation of a method for fixing a variation of the guide to the target site.

FIGS. 17a, 17b and 17c are front, side and top close-up views, respectively, of a variation of a method for fixing a variation of the guide to the target site.

FIGS. 18a and 18b illustrate front and top views, respectively, of a guide having an adhesive layer placed over the guide and skin to facilitate fixing of the guide relative to the target site.

### DETAILED DESCRIPTION OF THE INVENTION

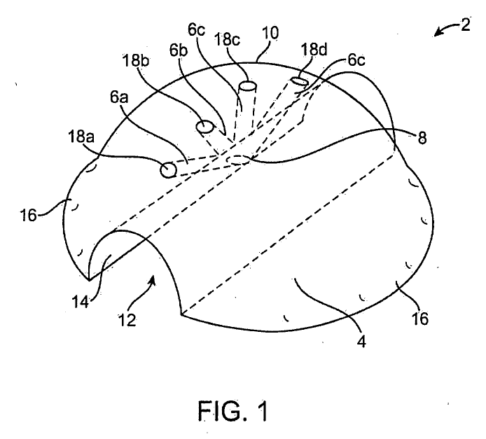

FIG. 1 illustrates a variation of a guide 2 that can be used, for example, to direct one or more surgical devices to be inserted through a single hole in tissue. The guide 2 can be used to minimize tissue damage during procedures that otherwise benefit from multiple tool entries through tissue at different angles and/or different adjacent locations.

The guide 2 can have a guide body 4 which can be substantially rigid or flexible. The guide body 4 can be made from a polymer, metal, or combinations thereof, and can include a crown 10 which may have a hemispherical configuration. The guide body 4 may also define a bone seat 12, such as a channel or groove, which can be configured to receive or otherwise seat on or adjacent to a target bone to be aspirated. The bone seat 12 can have a curved or an arcuate configuration formed by a seat wall 14, such as a cylindrical or semi-cylindrical configuration which extends along the length of the guide 2. Although the bone seat 12 need not extend along the entire length of the guide 2, the longer the seat 12 the greater the contact area against the target bone and subsequently the greater the stability of the guide 2 relative to the bone during a procedure. The cross sectional shape of the bone seat 12 can be varied in a number of shapes, such as circular, semi-circular, oval, semi-oval configuration, etc. or combinations thereof. The guide body 4 can also have a lip 16 extending radially around the perimeter of the guide body 4 to further enhance stability of the guide body 4 when placed against the patient body.

Within the guide body 4, one or more aspiration or guide channels 6, for example, a first, second, third and fourth aspiration channels 6a, 6b, 6c and 6d may be defined such that each of the aspiration channels 6 converge at a single exit port 8 which opens through the seat wall 14 into the bone seat 12. The first, second, third and fourth aspiration channels 6a, 6b, 6c, 6c and 6d can respectively have a first, second, third and fourth entry ports 18a, 18b, 18c, 18d which open along the crown 10 and each extend through guide 2 to the common exit port 8.

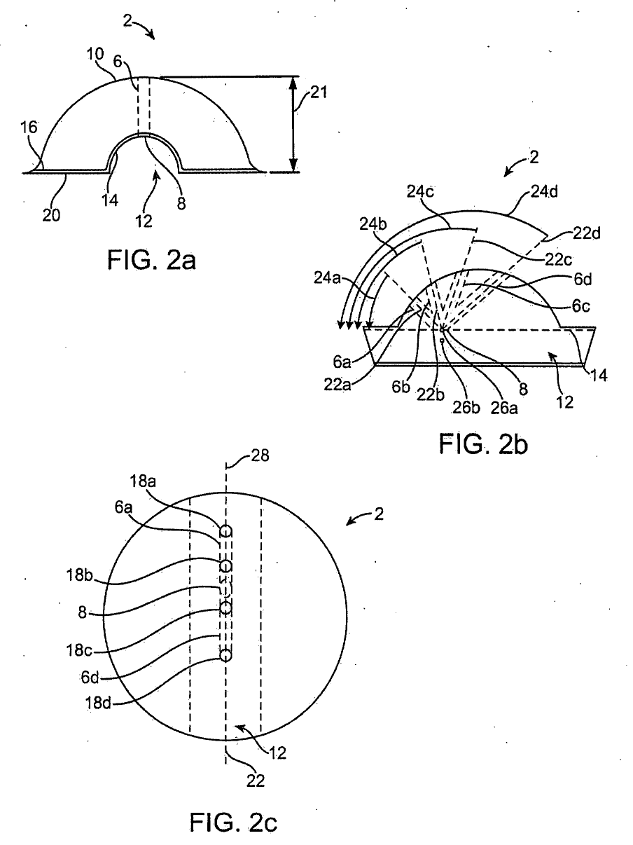

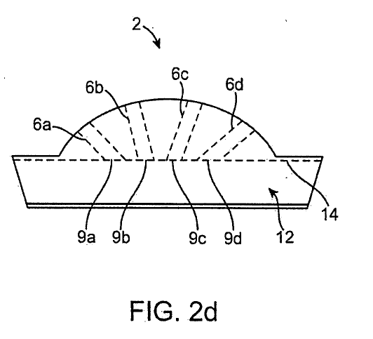

FIGS. 2a, 2b and 2c illustrate that the guide 2 can have a base 20 which may be substantially flat, as shown, or which can be curved in various configurations to approximate the tissue surface upon which guide 2 may be placed against. The base 20 can extend away from the sides of the bone seat 12 and can be made from a separate material than the remainder of the guide body 4, if desired. For example, the base 20 can be made from a relatively more rigid material than the guide body 4.

The guide 2 can have a guide height 21 which may vary depending upon the region of the body over which guide 2 is positioned upon and may generally range from about 10 mm (0.4 in.) to about 60 mm (2.4 in.), for example about 21 mm (0.84 in.) or about 50 mm (2 in.). The aspiration channels 6 can each have an aspiration channel longitudinal axis 22, e.g., as shown, the first, second, third and fourth aspiration channels 6a, 6b, 6e, 6c and 6d can respectively have a first, second, third and fourth aspiration channel longitudinal axis 22a, 22b, 22c and 22d. The aspiration channel longitudinal axes 22 can be at various angles from the plane defined by the top of the bone seat 12. For instance, as shown in FIG. 2b, the first, second, third and fourth aspiration channel longitudinal axes 22a, 22b, 22c and 22d can respectively be at a first, second, third and fourth aspiration channel angle 24a, 24b, 24c and 24d with respect to the plane defined by the top of the bone seat 12. For example, the first, second, third and fourth aspiration channel angles 24a, 24b, 24c and 24d can respectively be about 35°, about 50°, about 95°, and about 120°. In another variation, the first, second, third and fourth aspiration channel angles 24a, 24b, 24c and 24d can respectively be about 30°, about 45°, about 65°, and about 90°. The aspiration channel angles 24 can remain fixed or constant during use but may be varied depending upon the region of the body accessed.

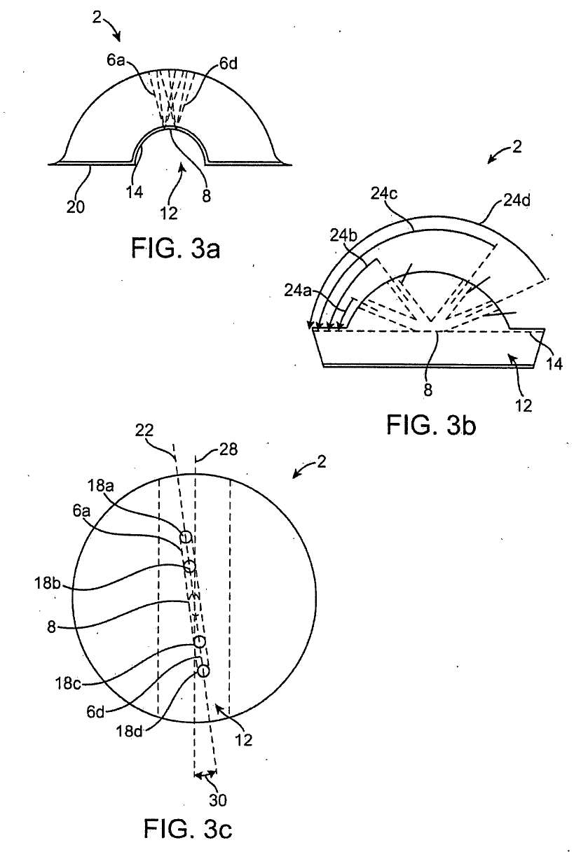

The first, second, third and fourth aspiration channel longitudinal axes 22a, 22b, 22c and 22d can be unevenly spaced from each other, as shown in FIG. 2b, or evenly spaced from each other, as shown in FIG. 3b. Moreover, the exit port 8 can be configured to be the same size and/or shape as the entry ports 18. As shown in FIG. 2c, the exit port 8 can be oblong, for example with a major axis in the direction of the aspiration channel longitudinal axes 22. The exit port 8 can be larger than the entry ports 18.

FIG. 2b illustrates that all or some of the aspiration channel longitudinal axes 22 can substantially intersect at a common, target point 26. As shown, a first target point 26a can be coincident within the exit port 8 or aligned with the top of the bone seat 12. Alternatively, the aspiration channel longitudinal axes 22 can converge at a second target point 26b at a point within or distally below the bone seat 12. For example, the second target point 26b can be located within the location of the cancellous bone marrow when the guide 2 is in use.

As shown in FIG. 2c, the aspiration channel longitudinal axes 22 can substantially align with a bone seat longitudinal axis 28 such that when bone seat 12 is aligned and positioned upon a bone to be accessed, such as along the iliac crest, the introduction of a trocar and/or bone marrow extraction device within any of the aspiration channels will initially introduce the instrument into the bone cavity within a single plane aligned with the bone.

Another variation of the guide 2 is illustrated in the side view of FIG. 2d which shows aspiration channels 6a, 6b, 6c, 6d angled with respect to the guide 2, as above. However, each channel may have an entry port in communication with its own respective exit port 9a, 9b, 9c, 9d rather than a single convergent exit port. Such a configuration allows for angled entry of an aspiration device into the underlying cavity through its own opening into the bone cavity.

FIGS. 3a, 3b and 3c illustrate a variation of the guide where the first, second, third and fourth aspiration channel longitudinal axes 22a, 22b, 22c and 22d can be evenly spaced from each other. For example, the first, second, third and fourth aspiration channel angles 24a, 24b, 24c and 24d can respectively be about 30°, about 70°, about 110°, and about 150° relative to the bone seat 12. In yet another variation, the aspiration channel longitudinal axes 22 can be substantially unaligned with the bone seat longitudinal axis 28. An aspiration channel-longitudinal offset angle 30 can exist between the aspiration channel longitudinal axes 22 and the bone seat longitudinal axis 28. The aspiration channel longitudinal offset angle 30 can be from about 0° to about 45°, more narrowly from about 1° to about 30°, for example about 5°.

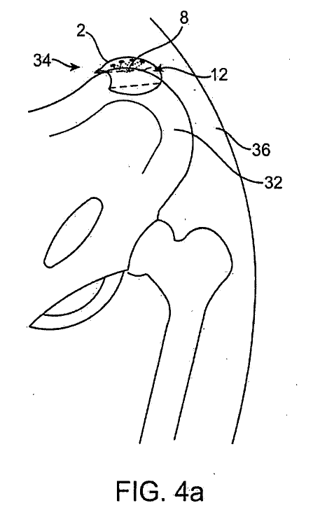

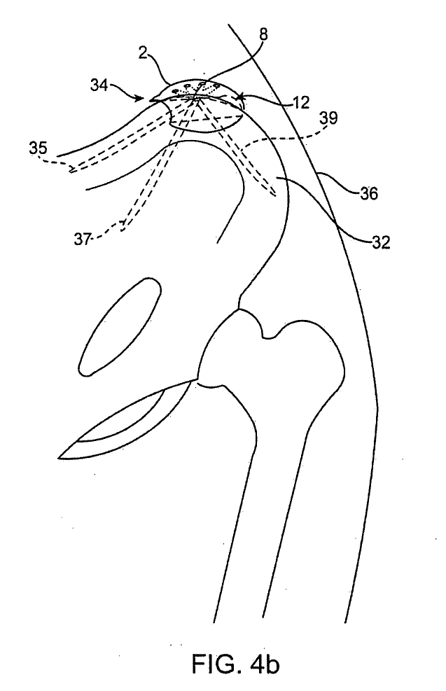

FIG. 4a illustrates one method of use where the guide 2 can be placed adjacent to the target bone, for example the guide 2 can be placed adjacent to the pelvis 32. For example, the bone seat 12 can be pressed onto the iliac crest 34, either directly upon the skin and soft tissue 36 left in place, or with the skin and soft tissue 36 cut and retracted out of the way of the bone seat. Accordingly, the exit port 8 can be placed in contact with the skin and soft tissue adjacent to the ileac crest 34 or directly against the ileac crest 34 itself.

In use, an aspiration cannula 306 (described below in greater detail) may be introduced into a first aspiration channel at a first angle through guide 2 to enable the device to sweep into the bone marrow matrix along a first path 35 within the bone cavity, as shown in FIG. 4b. Withdrawal and reintroduction of the aspiration cannula 306 into a second channel at a second angle through the guide 2 further enables the device to sweep into the bone marrow along a second path 37 to further harvest the bone marrow. The aspiration cannula may be further withdrawn and reintroduced into a third channel at a third angle through the guide 2 along yet a third path 39 to optimally harvest additional bone marrow. The process of withdrawal and reintroduction into the bone cavity under guidance by guide 2 at different angles allows for the aspiration cannula to sweep through the entire portion of the bone cavity.

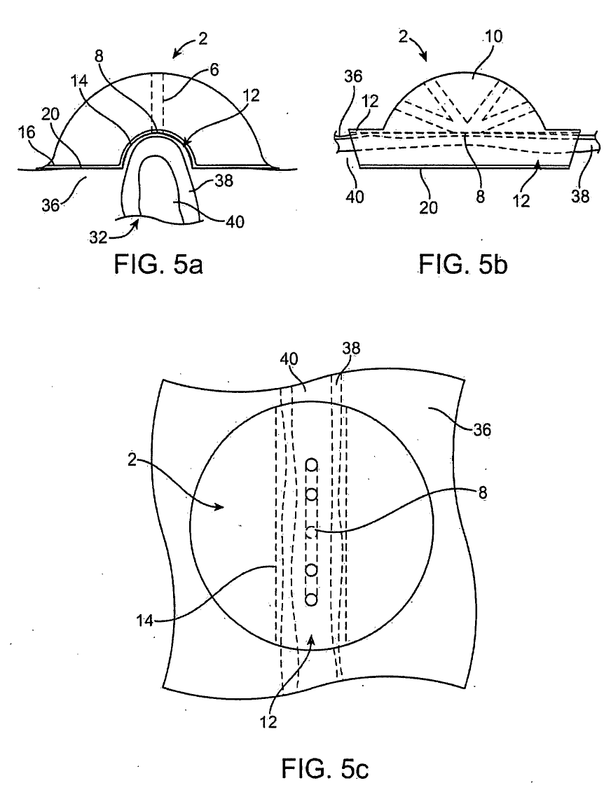

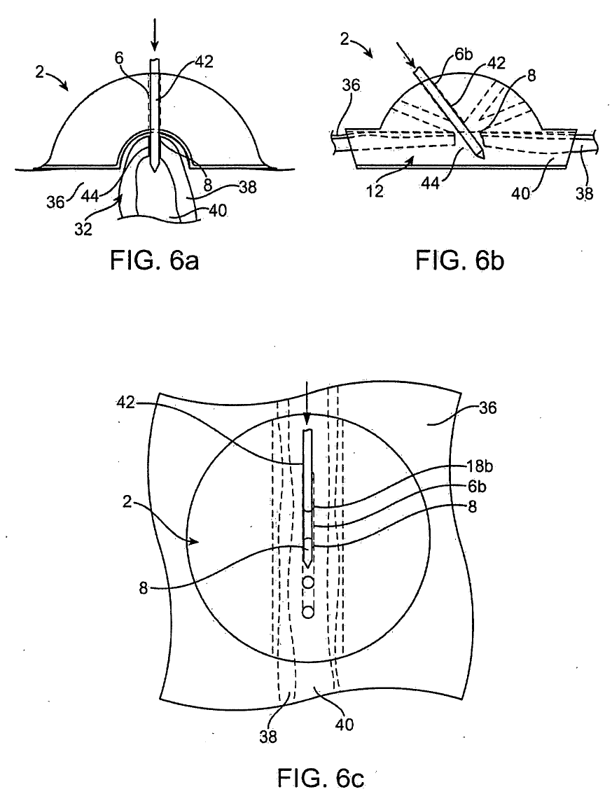

FIGS. 5a, 5b and 5c illustrate one use where the base 20 can be pressed as flush as possible with the soft tissue 36 against the skin of the patient. The exit port 8 can be positioned to provide close access through the soft tissue 36, cortical bone 38, and cancellous bone 40. As shown, bone seat 12 is placed against the bone 38 such that guide 2 is seated securely along the crest of the bone and the plane defined by the access channels 6 is aligned with a plane of the bone 38. FIGS. 6a, 6b and 6c illustrate that a hole can be bored in the soft tissue 36 and/or cortical bone 38 to access the cancellous bone 40. A bore 42 can be inserted through any of the aspiration channels 6 (shown as second aspiration channel 6b), as shown by arrow. The bore 42 can have a distal tip configured to cut through tissue, such as a sharpened and/or threaded tip such that the distal end of the bore 42 can be pushed and/or rotated out of the exit port 8 and through the adjacent soft tissue 36 and cortical bone 38 to create a cortical access channel 44 through the soft tissue 36 and/or the cortical bone 38 to create a substantially unobstructed path from the exit port 8 to the cancellous bone 40.

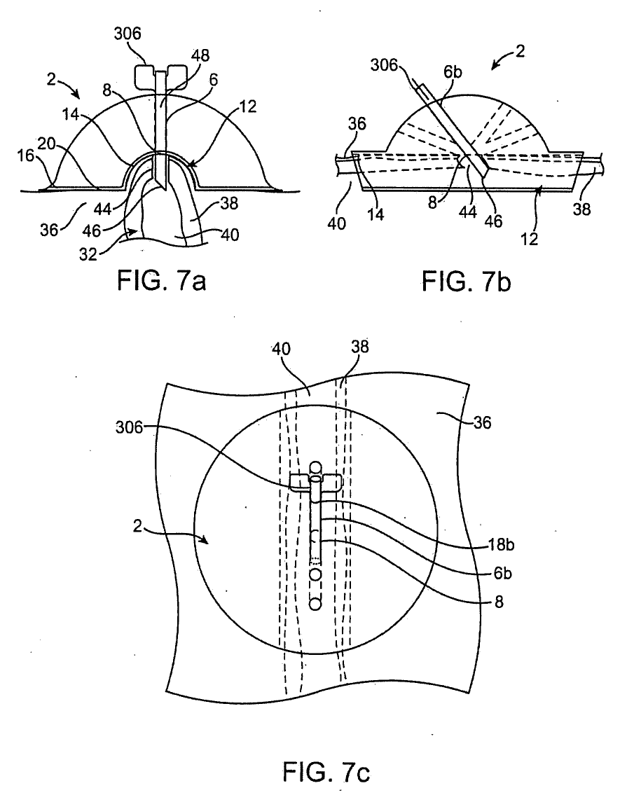

FIGS. 7a, 7b and 7c illustrate an access trocar 306 inserted in any of the aspiration channels 6 (shown as second aspiration channel 6b) where the trocar 306 has a sharpened or atraumatic trocar tip 46. The trocar tip 46 can be configured to cut through the soft tissue 36 and/or cortical bone 38. Alternatively, the trocar tip 46 can be configured to inhibit damage to soft tissue 36 and/or cortical bone 38, for example by being rounded or blunted. The trocar 306 can also have a hollow trocar channel 48 passing longitudinally through the trocar 306 and a length which can be rigid or flexible.

The trocar 306 can be passed through an existing cortical access channel 44 (e.g., created by a bore 42 or other device) or the trocar 306 can create a new cortical access channel 44 when the trocar 306 is inserted through the guide 2. The trocar 306 and/or bore 42 used to create the cortical access channel 44 can be manipulated, and/or the guide 2 can be supplementally manipulated (e.g., shaking, rotating, or “working” in the plane of FIG. 6b or 7b), to allow the trocar 306 and/or bore 42 to expand the cortical access channel 44 to a larger configuration than would have been created without manipulation of the trocar 306 and/or bore 42 and/or guide 2, for example by allowing for easier access to the cancellous bone 40. The trocar 306 and/or bore 42 and/or guide 2 can also be manipulated to make a smaller cortical access channel 44 and thus cause less tissue damage.

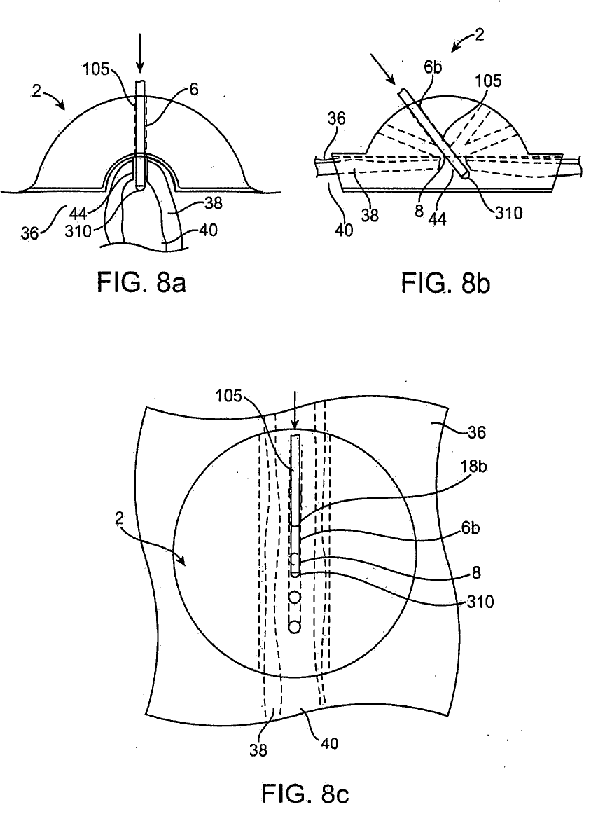

FIGS. 8a, 8b and 8c illustrate an example where a tissue disruption and aspiration device can be inserted into the cancellous bone marrow, either through a trocar channel 48 initially inserted through the guide 2 and left within the aspiration channel 6b (as shown in FIGS. 7a-7c) or directly through the guide 2 and through an access channel 44 created by a boring instrument or a trocar which has been removed from the guide 2. (The trocar is not shown in FIGS. 8a-8c for clarity of illustration). For example, the distal end of the aspiration cannula 105 of an aspiration device can be inserted through any of the aspiration channels 6 (shown as second aspiration channel 6b) and positioned into the cancellous bone 40.

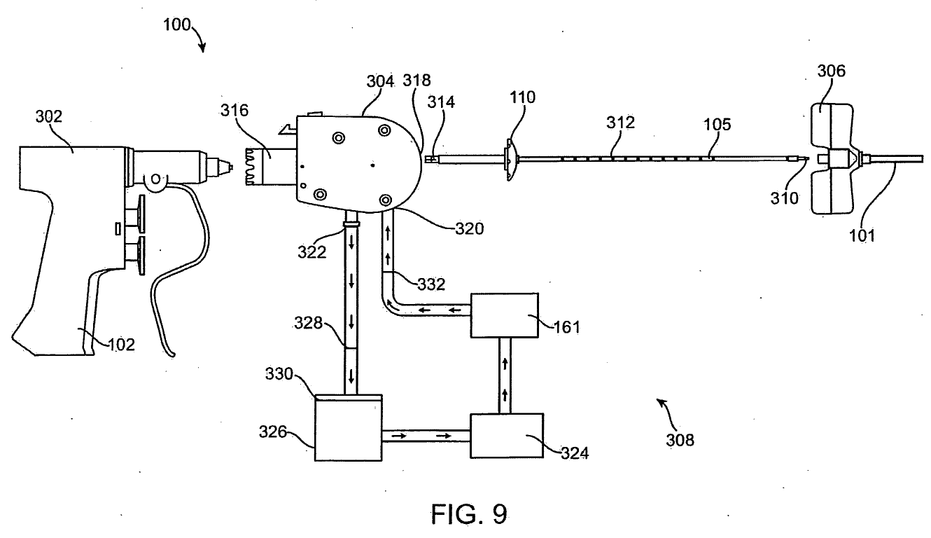

FIG. 9 illustrates an example of a tissue disruption and aspiration device 100 which can be used to aspirate and collect body tissue from within an enclosed body space in vivo or in vitro (also referred to as an “aspiration device” herein). Such an aspiration device, and aspiration catheters 105 and associated systems and elements, are described in further detail in U.S. patent application Ser. No. 11/750,287 filed May 17, 2007 as well as Ser. No. 10/454,846 filed Jun. 4, 2003, each of which is incorporated herein by reference in its entirety. As illustrated, the aspiration cannula 105 can have a curved member, such as whisk 310 which can be used to disrupt and aspirate cancellous bone 40. For example, the aspiration cannula 105 can be rotated and/or translated to and from different depths of the cancellous bone 40 to disrupt the matrix of the cancellous bone marrow. The marrow can be irrigated (e.g., via the aspiration cannula 105), for example with saline, and the marrow can be aspirated, for example caused by negative pressure exerted through the aspiration cannula 105. Moreover, the aspiration device 100 can alternatively have a drill 302, a connector and aspiration assembly 304, an aspiration cannula 105, an access trocar 306, and one or more fluid circuits 308.

The aspiration cannula 105 can attach to the connector 304 and/or drill 302 for ease of holding and operation such that the aspiration cannula 105 is in mechanical communication with the drill 302. The aspiration cannula 105 can be configured to be flexible or rigid and it may also include indentations, ridges, rings, visualization markers 312, or combinations thereof, for example to alter the flexibility of the aspiration cannula 105 along the entire length or a portion of the length of the aspiration cannula 105. The visualization markers 312 can be optionally radio-opaque and/or echogenic.

The aspiration cannula 105 may further include a rotational interface 314 configured to rotationally attach or couple to the connector 304 and/or the drill 302 for transmitting the rotational torque from the drill 302 to the cannula 105. The aspiration cannula 105 can further include a guard and/or a squash plate 110 to prevent over-insertion of the aspiration cannula into the connector 304 and/or the drill 302. The guard can non-rotationally attach to the connector 304 and/or the drill 302 such that during use, the guard can remain rotationally constant. The guard may further cover a gap between the aspirant cannula 105 and the connector 304 and/or drill 302, for example, to prevent the operator from pinching his/her hands in the device 100 while the aspirant cannula 105 is rotating.

The aspirant cannula 105 can further include one or more control wires along the length of the aspirant cannula 105. The squash plate 110 can be attached to the control wires such that the squash plate 110 can be manipulated by hand and/or by the connector 304 and/or by the drill 302 to steer, bend, flex, or combinations thereof, the distal end of the aspiration cannula 105.

The distal end of the aspiration cannula 105 can have a tissue disrupter such as a curved member, e.g., a whisk 310, which may be fixed, coupled, or otherwise integrated with the distal end of the aspiration cannula 105, as described in further detail below. The aspiration cannula 105 can facilitate aspiration and/or irrigation by defining one, two, or more lumens, for aspirating concurrently or subsequently to irrigating.

To provide an initial entry pathway into and through the cortical bone and into the medullary cavity, an access trocar 306 may be used which has an entry cannula 101 which defines an entry cannula channel that can pass through the length of the access trocar 306. The access trocar 306 can have one or more handles extending laterally and the entry cannula 101 can be configured to drive through cortical bone while guided by one of the channels 6 at an angle defined by the guide 2. Once the trocar 306 has been passed through the guide 2 and inserted and desirably positioned within the cortical bone creating an entry point, the aspiration cannula 105 may be passed through the entry cannula channel 101 and into the tissue matrix; accordingly, the channel 101 has a diameter which can reasonably accommodate the outer diameter of the aspiration cannula 105.

The connector and aspiration assembly 304 can have a drill interface 316 which mechanically couples the drill 302 and the connector 304 to one another via a removable interface which allows the drill interface 316 to couple and de-couple from the drill 302 itself. The connector and aspiration assembly 304 and/or the drill 302 can additionally include a mechanical transmission, for example, to increase and/or decrease the transmitted torque or speed from the drill 302 to the cannula 105. The connector and aspiration assembly 304 and/or the drill 302 can further include a governor, for example, to limit the rotational speed of the drill 302 transmitted to the aspiration cannula 105. Such a governor can be configured to be electronic, electromechanical, or mechanical in nature (e.g., as a resistor, slip-clutch, etc.) or combinations thereof. The maximum rotational speed of the aspiration cannula 105 can be from about 30 rpm to about 160 rpm, for example about 120 rpm.

The connector and aspiration assembly 304 can be further configured to direct and/or control aspiration and/or irrigation between the fluid circuit 308 and the first and/or second lumen of the aspiration cannula 105. The connector and aspiration assembly 304 can removably attach to the aspiration cannula 105 at a cannula port 318 and the connector and aspiration assembly 304 can further include an irrigation port 320 and/or aspiration port 322, each of which can be configured to be removably attached to fluid lines. The connector and aspiration assembly 304 can be configured to place the irrigation port 320 in fluid communication with a lumen in the aspiration cannula 105, for example a first lumen. The connector and aspiration assembly 304 can be further configured to place the aspiration port 322 in fluid communication with a lumen in the aspiration cannula 105, for example a second lumen, or the same lumen the irrigation port 320 is in fluid communication with.

The fluid circuit 308 can further include a pump 324 which is in fluid communication with an irrigant reservoir 161 and/or an aspirant reservoir 326. The irrigant reservoir 161 can have an irrigant, for example, saline solution. The pump 324 can deliver positive fluid pressure, as shown by arrows, to the irrigant reservoir 161 while also providing negative fluid pressure (i.e., suction), as shown by arrows, to the aspirant reservoir 326. The pump 324 can also be configured to reverse direction, i.e., providing negative pressure to the irrigant reservoir 161, and positive fluid pressure to the aspirant reservoir 326, for example, during cleaning to backwash the fluid system or to perfuse fluid into the tissue matrix to facilitate aspiration of the disrupted tissue. In this case, the irrigant perfusion rate can be, for example, from about 1 to 2 cc/min to about 30 cc/min.

An optional first aspiration filter 328 can be positioned in the flow between the aspiration port 322 and the aspirant reservoir 326 while an additional optional second aspiration filter 330 can be positioned in the aspirant reservoir 326, e.g., near the inlet port. An optional irrigation filter 332 can also be positioned between the irrigant reservoir 161 and the irrigation port 320. The first aspiration filter 328 and/or the second aspiration filter 330 can have pore sizes about 10 μm. While filters are shown positioned within the fluid lines or reservoirs, filters may alternatively be positioned within the cannula 105 itself, e.g., near or at the distal tip, for filtering out undesirable debris during aspiration such that the debris is prevented from passing through the cannula 105 and/or connector and aspiration assembly 304.

The drill 302, having a handle 102 and controls 103, can include any number of drills which are available for surgical purposes as interface 116 may be configured with a standard interface to couple and de-couple from any conventional drill interface. Examples of such drills 302 may include, for example, drills from DePuy Mitek, Inc. (Raynham, Mass.), Aesculap, Inc. (Center Valley, Pa.), Universal Driver or C.O.R.E. Micro Drill, Impaction Drill, Universal Series Drill (e.g., UHT Drill, U Drill), or Saber Drill commercially available from Stryker Corp. (Kalamazoo, Mich.), etc.

The aspirant reservoir 326 and the irrigant reservoir 161 integrated and/or attached to one another. As further shown, drill 302 can be engaged to connector and aspiration assembly 304.

The aspiration cannula 105 can have a degree of flexibility and/or curvature allowing the aspiration cannula 105 to follow the cavity (e.g., the intramedullary bone marrow space of the ileac or femur bone). The aspiration cannula can have an ultrasound transducer device at the distal tip 130 of the aspiration cannula 105, for example to visualize the cavity (e.g., define the width of the cavity).

As the aspiration catheter 105 is introduced into the body cavity, negative pressure can be initiated through the catheter 105, using a syringe or powered negative pressure device (e.g., pump), as the catheter 105 is advanced into the cavity. Negative pressure through the catheter 105 may be maintained while also withdrawing the device through the cavity. Alternatively, once the aspiration catheter 105 is fully introduced into the body cavity, the negative pressure can be initiated. As bone marrow is aspirated, the aspiration cannula 105 can be slowly withdrawn, with aspiration continuing as the aspiration cannula 105 is withdrawn. If sufficient amount of bone marrow is aspirated, the aspiration process is complete. Otherwise, after withdrawal of aspiration cannula 105, the curvature and/or directionality of the aspiration cannula 105 can be adjusted, and the aspiration cannula 105 can be redirected through the entry into the bone marrow space and manipulated to follow a different path through the space and aspirating more bone marrow. This process can be repeated for example 3-4 times, resulting in its aspiration of bone marrow from the majority of the bore marrow space (for example the ileac crest). This process can be repeated on both sides of the body or otherwise at multiple locations along the ileac crest or other bone target site as needed.

The access guide enables the aspiration catheter 105 to be introduced and guided into multiple regions within the body cavity through a single access port or entry. Moreover, the predetermined angles defined through the guide body also enables the aspiration catheter 105 to be guided to target specific regions within the body cavity, particularly along the iliac crest, which may provide the richest source and highest concentration of stem cells for harvesting.

Stem cells may be utilized to regenerate or improve function of damaged myocardium following a myocardial infarction, and may be useful in treating and preventing congestive heart failure. For example; a patient who has recently been diagnosed with a significant myocardial infarction and is brought to the catheterization suite, where interventional cardiologists perform angioplasty to open up a blocked coronary artery. Before, during or after the angioplasty procedure, a significant volume of bone marrow would be harvested. The bone marrow could be rapidly processed to enrich for hematopoietic stem cells or other populations or fraction of cells contained within bone marrow. These cells would then be delivered via catheter of other delivery device to the region of the heart which has undergone infarction and injury or death secondary to acute cardiac ischemia or other acute or chronic insults to the myocardial tissue. The delivered bone marrow or stem cell component contributes to regeneration of the myocardium or otherwise acts to improve cardiac function in the area of the infarct and leads to improved cardiac function and patient functional status and mortality. Optionally, marrow could be harvested separately from the initial cardiac catheterization procedure (for example 7 days after the MI, and in a separate procedure, stem cells or marrow enriched for stem cells could be delivered by any number of delivery mechanisms, for example by intracoronary or intramuscular injection. Use of a minimally invasive harvest device 100 would facilitate ease of harvest in patients who may be critically ill and not able to easily tolerate traditional marrow harvest procedures. In addition, minimally invasive harvesting of marrow has a role in intraoperative bone marrow harvesting for orthopedic applications.

As described above, there is the option of utilizing one or more aspiration cannulae 105 with preset or modifiable degrees of curvature and/or length and/or diameter and/or flexibility to adapt to different individual patients' anatomy and degree of ileac or other bone anatomy. Aspirated bone marrow can go directly into a bone marrow reservoir (e.g., the aspirant reservoir) or container through a closed system for initial storage and/or follow on manipulation, such as filtering, stem cell enrichment, or other follow on manipulation or treatment of bone marrow.

The apparatus and method shown herein provide many advantages for rapid aspiration and collection of body tissue from within an enclosed space. The directional control of the aspiration cannula by the operator enables the cannula to directly contact more of the marrow space and thereby aspirate a bone marrow that is more concentrated with stem cells than that available in the prior art. In addition, the harvest performed with the apparatus shown herein proceeds faster than prior art harvesting with a trocar since only one access point is required on each side of the body and less total volume of material is extracted. Finally, the procedure outlined above requires less time and reduced support personnel, thereby reducing costs for a procedure for harvesting bone marrow and/or tissue.

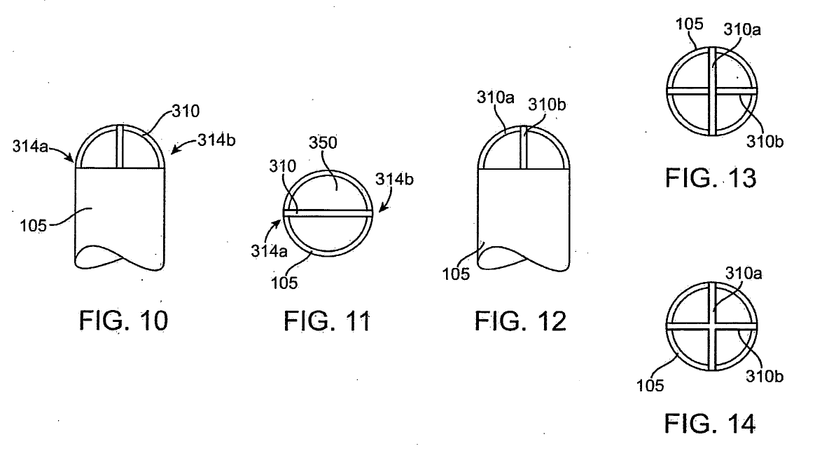

FIGS. 10 and 11 illustrate additional variations of the aspiration cannula 105 incorporating a tissue disruptor end effector configured in this variation as a whisk 310, as mentioned above. The whisk 310 can have a whisk first end 314a and a whisk second end 314b which can be attached to, or integral with, the distal end of the aspiration cannula 105. While the whisk 310 is illustrated as having a semi-circular or looped configuration, it may be configured in any number of shapes so long as clearance between the whisk 310 and cannula opening 350 is provided to allow for entry of the disrupted tissue therethrough. The whisk 310 can be resilient or deformable or alternatively flexible or rigid. The whisk 310 is also preferably rigid enough to disrupt cancellous bone yet flexible enough so as to not penetrate cortical bone during normal use.

FIG. 12 illustrates another variation with the cannula 105 utilizing two or more whisks 310a and 310b. The first and second ends of the whisks 310a, 310b can be attached to and/or integral with the distal end of the cannula 105. FIG. 13 illustrates an end view of a variation where that the first whisk 310a can be non-integral, unattached, or unconnected from the second whisk 310b while FIG. 14 illustrates likewise illustrates an end view of another variation where the first whisk 310a can be integral, coupled, or otherwise attached with the second whisk 310b.

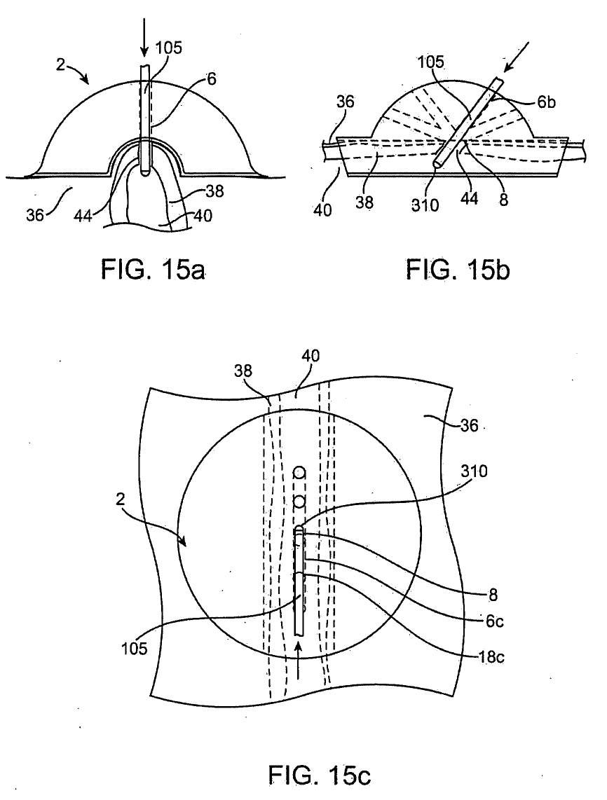

FIGS. 15a, 15b, and 15c illustrate a use where the aspiration cannula 105 and/or the trocar 306 (the trocar 306 is not shown for clarity of illustration) can be removed from the second aspiration channel 6b. If the trocar 306 is used, the trocar 306 can be inserted through the third aspiration channel 6c and out the exit port 8. If the existing cortical access channel 44 does not already provide a clear path from the exit port 8 to the cancellous bone 40 along the angle of the third aspiration channel longitudinal axis 22c, the trocar 306 can expand the cortical access channel 44 along third aspiration channel longitudinal axis 22c to provide substantially unobstructed access between the third aspiration channel 6c and the cancellous bone 40. The bore 42 can be used instead of, or in addition to, the trocar 306 to expand the cortical access channel 44.

The aspiration cannula 306 can be inserted through the third aspiration channel 6c and out the exit port 8. The aspiration cannula 306 can be inserted through the trocar channel 48 with the trocar 306 positioned in the third aspiration channel 6c, as shown in FIGS. 7a-7c (but through the third aspiration channel 6c). Alternatively, the aspiration cannula 105 can be inserted through the third aspiration channel 6c without the trocar 306.

The aspiration cannula 306 can then further aspiration and/or irrigation the cancellous bone 40 and/or disrupt the cancellous bone matrix. The aspiration cannula 105 and/or the trocar 306 and/or the bore 42 can be removed from the third aspiration channel and inserted in the first and/or second and/or fourth aspiration channels 6a and/or 6b and/or 6d. Additional aspiration and/or irrigation and/or disruption of the cancellous bone 40 can then be performed by the aspiration cannula 105. Furthermore, the bore 42 can be deployed through the trocar channel 48. The guide 2 can be removed when the aspiration of cancellous bone 40 is complete. The guide ˜2 can also be repositioned if aspirating cancellous bone from a different position is desired.

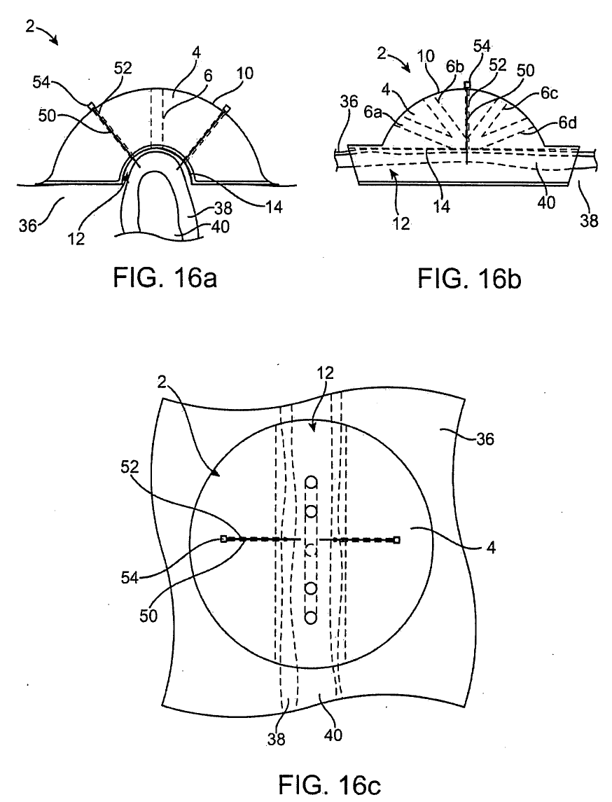

FIGS. 16a, 16b, and 16c illustrate an example where the guide 2 can be fixed to the target site using, for example, one, two, or more fixation pin channels 50 passed through guide body 4 and into the underlying bone to temporarily anchor the guide 2 thereto for enhanced stability during an access procedure. The fixation pin channels 50 can open at a first end on the crown 10 and open at a second end on the seat wall 14. The fixation pin channels 50 can be evenly distributed around the guide 2.

Once the guide 2 is placed in position adjacent to the target site, one or more of the fixation pins 52 can be inserted through each fixation pin channel 50. The fixation pins 52 can be configured to pass through soft tissue 36 and cortical bone 38. Fixation pin heads 54 can be integral with or attached to the fixation pins 52 where the fixation pin heads 54 can be larger in diameter than the fixation pin channel 50 to prevent the fixation pin 52 from descending too far down the fixation pin channel 50 such that removal of the fixation pins 52 is difficult. The fixation pin head 54 can be a surface for the fixation pin 52 to be indirectly impacted, for example by a hammer or other impact tool. The fixation pin head 54 can be grabbed and pulled to remove the fixation pin 52 from the fixation pin channel 50. Moreover, the fixation pins 52 can be removed from the cortical bone 38 and the guide 2 when the aspiration of cancellous bone 40 is complete or if the guide 2 is to be repositioned.

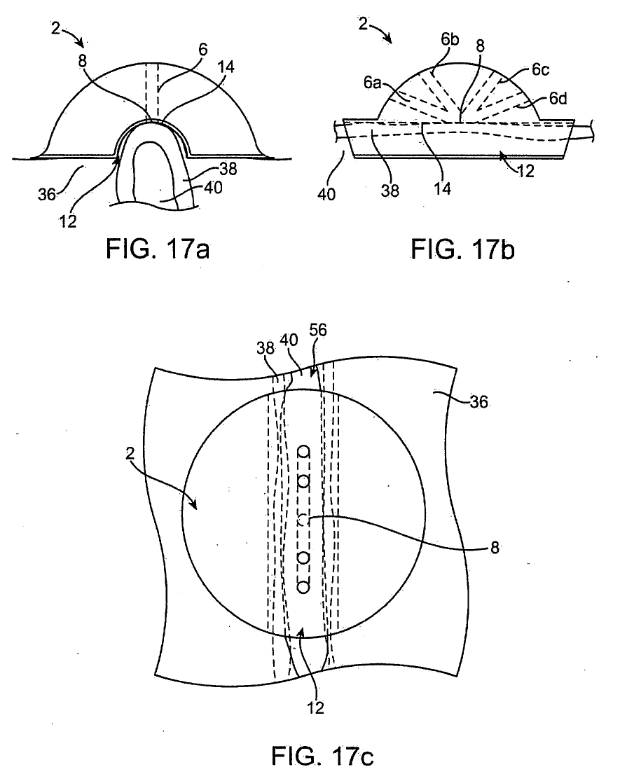

FIGS. 17a, 17b, and 17c illustrate an example where the soft tissue 36 can be cut and retracted away from the eventual site of the cortical access channel 44. A retracted area 56 can be created from where the soft tissue 36 has been retracted, substantially exposing the cortical bone 38. The soft tissue 36 can be retracted substantially away from just the location of the cortical access channel 44 and the exit port 8 (as shown), or entirely away from the location of the base 20 of the guide 2. The exit port 8 can be in substantially direct contact with cortical bone 38 such that the seat wall 14 is in direct contact with the surface of the cortical bone 38.

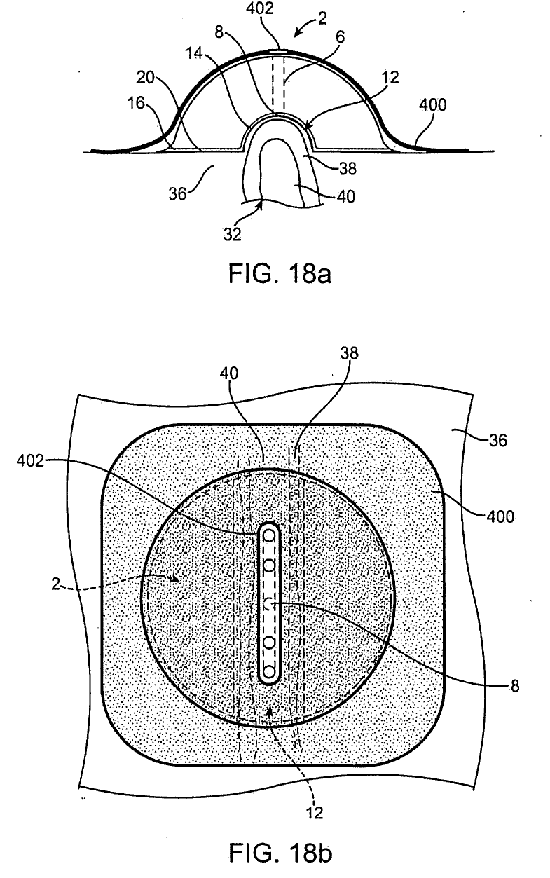

FIGS. 18a and 18b illustrate front and top views, respectively, of a guide 2 having an adhesive layer 400 placed directly over the guide 2 and skin to facilitate fixation of the guide 2 relative to the target site. As shown, layer 400 may comprise any number of adhesive-backed materials which may be placed entirely over the guide 2 as well as upon the surrounding tissue area. Layer 400 may also define an opening 402 through which the access channels may be accessed and through which the aspiration device may be introduced into the guide 2. Moreover, layer 400 may be configured into any number of shapes and sizes so long as the guide 2 and a portion of the surrounding tissue is covered by layer 400 to provide the stability sufficient for maintaining the position of the guide 2 against the tissue region of interest and to prevent the movement of the two relative to one another.

Although the use of the guide 2 is frequently shown and described for aspiration of cancellous bone, the harvesting of any tissue can be performed (e.g., tumor biopsy). The guide 2 can also be used to provide directional guidance for any elongated surgical tools at a variety of fixed angles through a single hole at the target site.

It is apparent to one skilled in the art that various changes and modifications can be made to this disclosure, and equivalents employed, without departing from the spirit and scope of the invention. Elements shown with any variation are exemplary for the specific variation and can be used on or in combination with any other variation within this disclosure.

What is claimed is:

1. A guide device for deploying surgical tools to target sites comprising:
a guide body; and
at least one channel angled to guide an instrument to a first target region within a body cavity.

2. The device of claim 1, wherein the at least one channel comprises a first guide channel at a first angle with respect to the remainder of the guide.

3. The device of claim 2, further comprising a second guide channel at a second angle with respect to the remainder of the guide, wherein each channel has an angle different from one another.

4. The device of claim 3, wherein the first guide channel has a first entrance port and a first exit port, and wherein the second guide channel has a second entrance port and a second exit port coincident with the first exit port.

5. The device of claim 1, wherein the guide body has a seat configuration to seat on or adjacent to the target site.

6. The device of claim 5, wherein the seat has an arcuate cross-sectional configuration.

7. The device of claim 5, wherein the seat has a semi-circular cross-sectional configuration.

8. The device of claim 1, wherein the guide body is substantially rigid.

9. The device of claim 3, wherein the first guide channel is angled at the first angle of 35° relative to the guide body.

10. The device of claim 3, wherein the second guide channel is angled at the second angle of 50° relative to the guide body.

11. The device of claim 3, further comprising a third guide channel having a third angle with respect to the remainder of the guide.

12. The device of claim 11, wherein the third guide channel is angled at the third angle of 95° relative to the guide body.

13. The device of claim 11, further comprising a fourth guide channel having a fourth angle with respect to the remainder of the guide.

14. The device of claim 13, wherein the fourth guide channel is angled at the fourth angle of 120° relative to the guide body.

15. The device of claim 1, further comprising at least one fixation mechanism for maintaining a position of the guide body relative to the target region

16. The device of claim 15, wherein the fixation mechanism comprises at least one fixation pin.

17. The device of claim 15, wherein the fixation mechanism comprises an adhesive layer for placement upon the guide body and over at least a portion of the target region.

18. A guide device for deploying surgical tools to target sites comprising:
a guide body;
a first guide channel having a first entrance port and a first exit port; and
a second guide channel having a second entrance port and a second exit port substantially coincident with the first exit port.

19. The device of claim 18, wherein the guide body has a seat configuration to seat on or adjacent to the target site.

20. The device of claim 19, wherein the seat has an arcuate configuration.

21. The device of claim 19, wherein the seat has a semi-circular configuration.

22. The device of claim 18, wherein the guide body is substantially rigid.

23. The device of claim 18, wherein the first guide channel is angled at 35° relative to the guide body.

24. The device of claim 18, wherein the second guide channel is angled at 50° relative to the guide body.

25. The device of claim 18, further comprising a third guide channel having a third entrance port and a third exit port substantially coincident with the first exit port.

26. The device of claim 25, wherein the third guide channel is angled at 95° relative to the guide body.

27. The device of claim 25, further comprising a fourth guide channel having a fourth entrance port and a fourth exit port substantially coincident with the first exit port.

28. The device of claim 27, wherein the fourth guide channel is angled at 120° relative to the guide body.

29. A method for removing bone marrow from a target volume within a subject, comprising:
positioning a guide adjacent to the target volume, wherein the guide has a first channel and a second channel;
advancing a removal tool through the first channel and into the target volume at an entry port;
removing a first portion of the bone marrow with the removal tool;
withdrawing the removal tool from the target volume and the first channel; and
advancing the removal tool through the second channel and into the target volume at the entry port.

30. The method of claim 29, further comprising removing a second portion of the bone marrow with the removal tool.

31. The method of claim 29, wherein the advancing of the removal tool through the first channel comprises advancing the removal tool at a first angle relative to the guide, and wherein the advancing of the removal tool through the second channel comprises advancing the removal tool at a second angle relative to the guide.

32. The method of claim 31, wherein advancing the removal tool at a first angle comprises advancing the tool at an angle of 35° relative to the guide.

33. The method of claim 31, wherein advancing the removal tool at a second angle comprises advancing the tool at an angle of 50° relative to the guide.

34. The method of claim 29, further comprising fixing the guide to the subject.

35. The method of claim 29, further comprising disrupting a tissue matrix at the target site, and aspirating the disrupted tissue matrix.

36. The method of claim 29, wherein the target site comprises a medullary cavity of the subject.

37. The method of claim 29 wherein positioning comprises adhering the guide adjacent to the target volume to maintain a position of the guide with respect to the entry port.
